(19) United States
(12) Patent Application Publication
(10) Pub. No.: US 2009/0143668 A1
(43) Pub. Date: Jun. 4, 2009

(54) ENHANCEMENT OF MRI IMAGE CONTRAST BY COMBINING PRE- AND POST-CONTRAST RAW AND PHASE SPOILED IMAGE DATA

(76) Inventors: Steven E. Harms, Fayetteville, AR (US); Scott Spangenberg, Amherst, NH (US); Xiaole Hong, Acton, MA (US)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/328,481

(22) Filed: Dec. 4, 2008

Related U.S. Application Data
(60) Provisional application No. 60/992,208, filed on Dec. 4, 2007.

(51) Int. Cl. A61B 5/05; G06K 9/00

(52) U.S. Cl. 600/410; 382/131

(57) ABSTRACT

An MRI process and system image a volume of a sample in a magnetic field established by a biasing field magnet and an array of gradient magnet fields using a pulse sequence to obtain a response that is decoded into an image or images. A set of successive images is collected while the contrast associated with lesions and tumors is enhanced with a contrast agent. A non-spoiled reference image is acquired before the application of the contrast agent. The reference image is non-spoiled in that the pulse sequence for collecting a portion of the volume image is not randomized in phase in a manner that would reset the phase effects of a previous pulse sequence. At least one other one of the successive images collected using phase spoiling pulse sequences. The non-spoiled image data is registered with and subtracted from the successive images to enhance the appearance of selected compositions in the output image, such as the contrast agent and/or water to highlight lesions and cysts, or silicone from an implant, etc., which can be highlighted by color coding.

### FIELD

The invention relates to three dimensional volume imaging, especially medical magnetic resonance imaging using contrast agents.

### BACKGROUND

Medical magnetic resonance imaging (MRI) is a non-invasive technique that relies on the relaxation properties of nuclei when subjected to a steady state magnetic biasing field. The nuclei of atoms have magnetic moments that can be aligned by being subjected to a biasing magnetic field. Once aligned by the steady state magnetic biasing field, the nuclei can be excited by applying a radio frequency (RF) signal at the resonance frequency, known as the Larmor frequency, for a particular element or isotope. When excited at the Larmor frequency, the magnetic moments of the nuclei of the element or isotope are momentarily realigned.

Following the reorienting pulse, the nuclei relax over a period of time (T1) and return to their original alignment relative to the biasing field, B₀. The specific time period varies with the type of nuclei, the incident magnetic fields, and the amplitude of the excitation pulse. The phase-synchronized spins of a group of adjacent nuclei reinforce each other to produce a detectable spin echo signal at the resonance frequency. The spin echo signal can be resolved to determine the corresponding location in a volume, i.e., a voxel value. The spin echo of the nuclei attenuates over a period of time (T2) as an increasing number of nuclei fall out of phase with, and no longer reinforce, the other nuclei. The time period T2 is related to the type of nuclei, the bias and excitation conditions, as well as the temperature of the sample being imaged.

Being able to selectively produce an echo signal from the nuclei of a specific element enables the detection of the differences in tissue composition. For example, by exciting tissue at the resonance frequency of hydrogen, tissues with high concentrations of water (H₂O) produce a more robust response than tissues having low concentrations of water. Similarly, at a slightly different resonance frequency, it is possible to excite hydrogen that are concentrated in fatty tissues (e.g., lipids). Additionally, by modulating the strength of gradient magnetic fields over time, while applying a timed sequence of excitation pulses followed by signal reception intervals, a radio frequency response is produced at a given point in time. This radio frequency response can be spatially addressed and uniquely associated with a point in a volume. Fourier transforms are then used to resolve the radio frequency response to a localized point.

One object of medical MRI is to collect data values to distinguish between different types of tissue by location. However, to distinguish between different tissue types, fine spatial and amplitude resolutions are needed to a minimum incremental volume that is pertinent to tissue structure. Imaging data can be represented by mapping different data amplitudes to points in two or three dimensions. The different amplitudes can be represented by mapping a range of amplitudes to a range of luminance (brightness) levels over a gray scale. The mapped data can be displayed in a graphical projection on a display screen. For example, an image of tissues adjacent to a theoretical slice through the tissue can be shown in two dimensions (2D).

In some applications, it may be preferable to display tissue types as opaque elements in a volume that are otherwise shown as substantially transparent. Displaying imaged features transparently or opaquely helps to reveal tissue structures, surface characteristics, and the like. The tissue structures are projected onto a two dimensional display screen, and anatomical features can be visualized by rotating the projection to view the projected volume from different perspectives. Various results are obtainable using different excitation pulse sequences to develop voxel values in three dimensions (3D), where the encoded value for each voxel represents a response of a particular element with respect to one or more parameters. The distinguishing parameters can be the amplitude of the RF emission at a resonance frequency, the rate of the fading away of the echo response, and other aspects that permit one element to be distinguished from another element and/or permit the assessment of the relative concentrations of elements at different locations.

The distinct responses are also useful in distinguishing between different types of tissue based on the relative concentrations of two or more elements. For example, magnetic resonance imaging may be used to distinguish between fat and muscle or between different tissue structures such as blood vessels or concentrations of edema or ischemia. A given tissue type can be highlighted in an image by varying the brightness, color, or opacity of the tissue. Alternatively, a given tissue type can be caused to appear dark or transparent to better emphasize a different tissue type or to reveal other tissue types that may be located behind the transparent tissue type in a projection of a volume. Such distinctions can also be visually presented in image slices through an opaque tissue volume.

An important application for the magnetic resonance imaging as described above is the diagnosis and treatment of breast cancer. By distinguishing tissue types, for example by distinguishing concentrations of fat from concentrations of water and thereby distinguishing between tissue types, the internal breast tissue structures, such as ducts and vasculature, can be more easily visualized. Fatty tissues can be rendered transparent or dark in a volume projection to highlight duct structures or to impart contrast to the image. The rendering of tissues as transparent or dark enables a practitioner to distinguish cysts from tumors, and so forth. Contrast agents can be introduced to improve the extent to which pertinent tissue types and tissue structures can be distinguished. For example, gadolinium-based contrast agents can be injected to enhance the contrast of particular tissue types and to limn the contours of blood vessels and other structures. Tissues can be distinguished with respect to the rate at which a perfused contrast agent washes out over time.

In certain NMR/MRI arrangements, the gradient magnetic fields are placed and modulated to image thin slices of tissue. The collected data for the respective pixels in each slice are associated as a stack of slices. The spatial resolution of volume elements (voxels) corresponds to the x-y resolution within a slice and the pitch spacing between successive slices. However, it is not necessary always to modulate the bias and gradient fields in an orthogonal x-y and stepped z-raster-like progression of slices. In a different technique, such as a spiral imaging technique exemplified by commonly owned U.S. Pat. Nos. 5,202,631, 5,304,931, and 5,415,163, incorporated by reference herein in their entireties, the fields are modulated to target a succession of k-space data in a spiral pattern. The data is collected in successive iterations, and the voxel resolution is related to the imaging time. It is desirable to collect an image quickly, but a fine image resolution and heavy contrast is also desirable and requires longer imaging passes. Therefore, it is generally necessary to reach a compromise between image collection time and image resolution and contrast.

Thus, an improved method and system for increasing the contrast features in an MRI image is desired.

### SUMMARY

It is an object of the present disclosure to improve the contrast between types of tissue represented in an MRI output image, particularly when using gadolinium-based contrast agents. This is accomplished by taking into account a preliminary reference image of a sample. The reference image voxel values are subtracted from corresponding values in one or more volume images of the sample taken later, especially after application of the contrast agent. The result is to provide higher contrast for particular features such as lesions and tumors, than would otherwise be found in the later images.

Another aspect of the disclosure is that the pre-contrast raw baseline images are collected using a pulse sequence that does not use preliminary de-phasing (“spoiling”), whereas the post-contrast images are collected using a pulse sequence that employs phase spoiling. The contribution of fluid rich tissue is decreased by RF spoiling in the pulse sequence. When the pre-contrast, non-spoiled image is subtracted from plural successive spoiled images collected after introduction of the contrast agent, the contribution of fluid tissues is disproportionately reduced. The effect is useful to enhance the contrast between relatively lower fluid density tissues such as lesions, the contrast of which is made relatively greater (these features are made brighter in a normalized image), versus higher fluid density tissues such as cysts, which are deemphasized. At the same time the effect also helps the practitioners to identify the fluid rich tissues such as cysts.

### BRIEF DESCRIPTION OF THE DRAWINGS

There are shown in the drawings certain illustrative embodiments of the present subject matter; however, it should be appreciated that the invention is not limited to the embodiments disclosed as examples and is capable of variations in keeping with the scope of the subject matter defined in the appended claims. In the drawings,

FIG. 1 is a perspective view of an exemplary nuclear magnetic resonance imaging system;

FIG. 2 is a block diagram illustrating the basic elements of the nuclear magnetic resonance imaging system shown in FIG. 1;

FIG. 3 is a schematic illustration showing the area of primary imaging linearity as appropriate for imaging the breasts;

FIG. 4 is an illustration showing an exemplary spiral data collection pattern for collecting voxel data values in a three dimensional volume;

FIG. 5 is a timing diagram demonstrating the relationship of excitation pulses and gradient modulation; and

FIG. 6 is a flow chart of an exemplary imaging sequence used by the system of FIG. 1.

### DETAILED DESCRIPTION

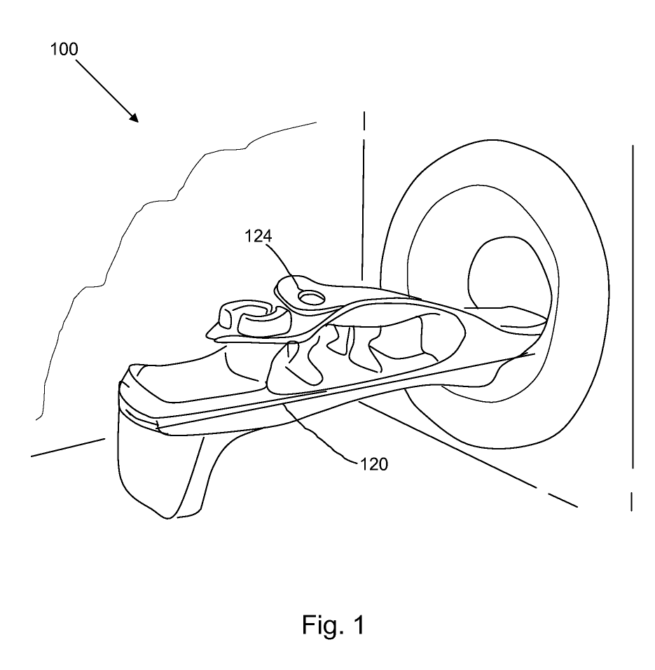

FIGS. 1-3 generally show the elements of a nuclear magnetic resonance imaging arrangement, in a configuration that is appropriate for imaging the human breasts. This configuration, operated with a spiral gradient sequence as explained below, is particularly useful in connection with screening and diagnostic operations for breast cancer; however, the system and method described herein are not limited to such applications.

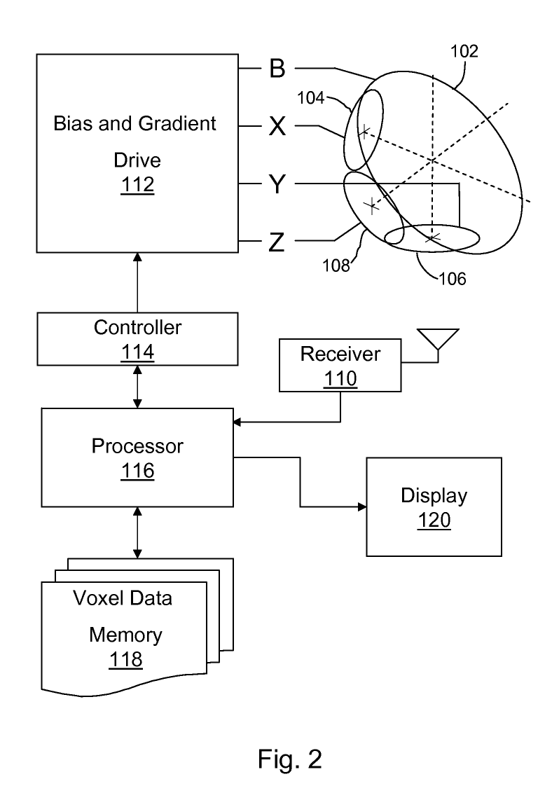

The system 100 comprises a set of electromagnets including a biasing coil 102 (shown in FIG. 2) for establishing a static magnetic biasing field, B₀, in a longitudinal direction with respect to a patient (not shown in FIGS. 1 and 2) lying on a table 122. Table 122 can be translated into the lumen of the biasing coil 102 to a position where the biasing magnetic field is substantially isometric. The patient lies prone, feet toward the coil 102, with breasts depending through openings 124 in the table 122 into an accessible zone. In certain procedures, the breasts may be held stable in a fixture (not shown) to facilitate biopsy procedures undertaken with the aid of positioning guidance from the imaging data obtained using the apparatus.

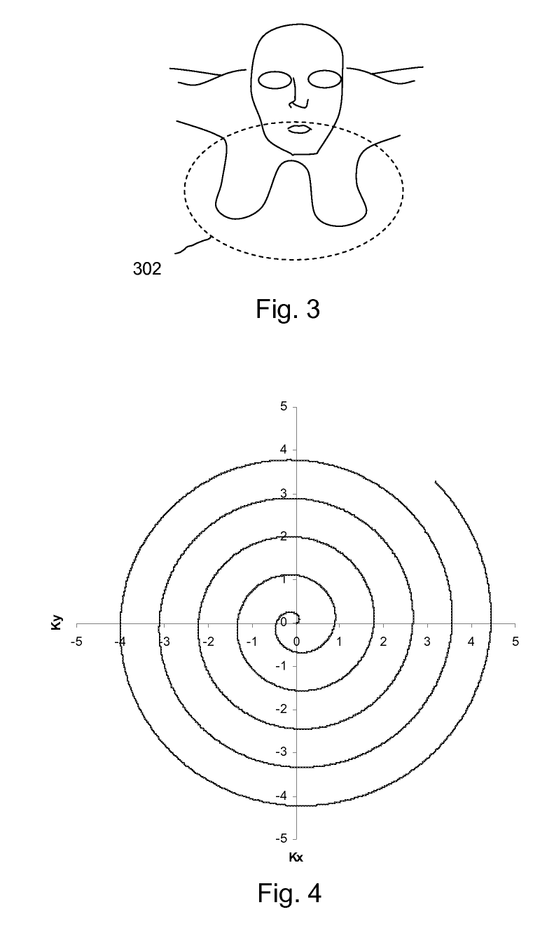

As shown in FIG. 2, biasing coil 102 is positioned to provide a static magnetic field in the longitudinal or z-direction. Additional coils 104,106 are positioned to apply magnetic field gradients in the orthogonal x- and y-directions, respectively. A phase-encoding coil 108 is positioned with an orientation parallel to that of the biasing coil 102 in the z-direction. In an embodiment appropriate for breast imaging, the apparatus is configured and dimensioned primarily to image a volume encompassing the breasts and the anterior thoracic area 302 of the patient, as shown by the dashed lines in FIG. 3.

As shown in FIG. 1, a controller 114 is coupled to a processor 116 and to an electric drive 112. Electric drive 112 is configured to apply a sequence of excitation and encoding pulses to the x- and y-gradient coils 104, 106, and to the phase-encoding z-gradient coil 108. Receiver 110 is configured to receive response signals from the excitation and encoding pulses and transmit the received signals to processor 116. Processor 116 is programmed to demodulate and decode the received signals and use Fourier Transforms to decode the signals, expressed as K-space data, into images that can be stored in memory or as files. The stored image data includes multi-dimensional arrays of which at least one data value is applicable to each volume element (voxel) in the imaged volume containing the patient's breasts. The image data may be presented by the processor 116 on a display 120 so that a practitioner can visualize internal breast tissue structures.

The processor 116 can apply various image processing steps to the voxel data in order to enhance the image. Without limitation, such steps can include enhancement of contrast by edge detection, threshold level discrimination, the application of pattern enhancement masks, image analysis transforms, and the like. According to one aspect, the processor 116 is arranged to collect plural images of the same volume before and after one or more processing steps. These images are applied to one another such that voxels in registry are added, subtracted, or subjected to thresholds and Boolean operations to enhance the contrast of an image.

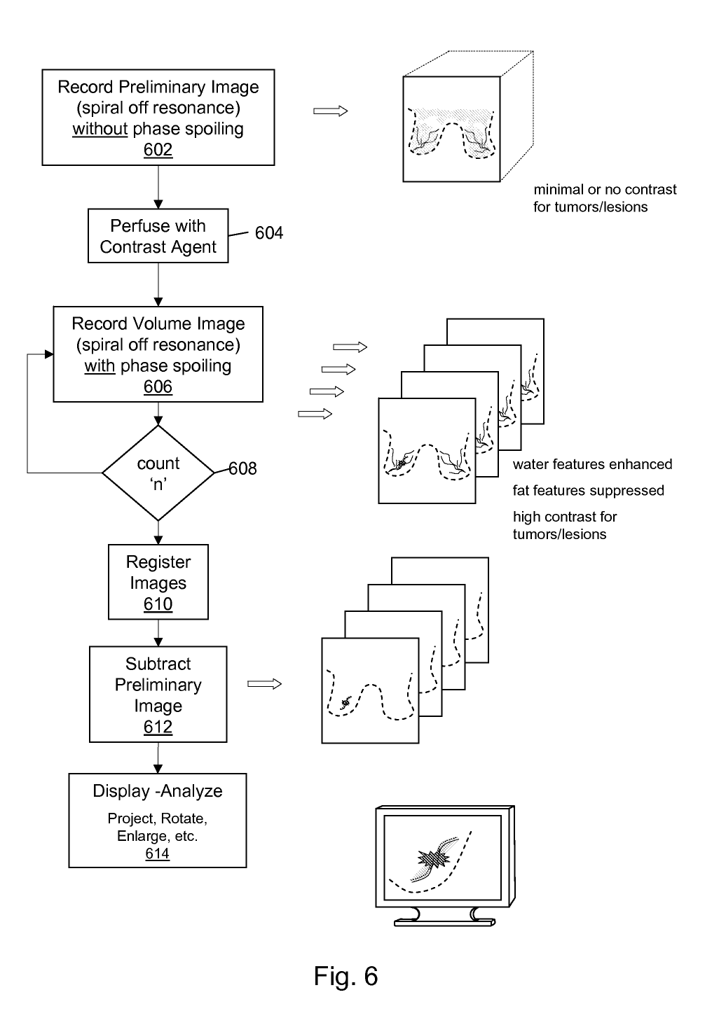

With reference to FIG. 6, an exemplary method for enhancing the contrast of an image is described. A set of baseline images are collected prior to the application of a contrast agent at block 602. These images can be obtained without the use of phase spoiling during the imaging sequence. Without the use of phase spoiling, the image tends to reveal concentrations of fluid, e.g., edema and cysts. At block 604, a contrast agent is perfused in the body of a patient and a series of ‘n’ images is then collected at blocks 606 and 608. The contrast agent may be a gadolinium-based contrast agent or other paramagnetic contrast agent that tends to concentrate in a lesion and display brightly in an image. By concentrating in a lesion, the contrast agent enhances the contrast of lesions in the collected image data. The post-contrast imaging passes include the use phase-spoiling to substantially randomize the phase conditions between the image data collection sequences.

Once all post-contrast imaging passes have been performed and the images are registered at block 610, the baseline images without phase-spoiling are subtracted from the phase-spoiled, contrast-enhanced images at block 612. The preliminary image may be subtracted from each of the images, or only a select group of the images depending on the desired contrast. In one arrangement, there is one set of N baseline images that are obtained before injection of the contrast agent. There are M sets of N images that are obtained post-contrast, each of which covers the same volume as the baseline set. Subtraction of images is performed such that a particular baseline image is subtracted from the corresponding image of a given post-contrast set, resulting in M sets of N subtraction images.)

As a result of the image subtraction, fluid in the tissue (edema, cysts, etc.) is darkened such that a projection of the high contrast lesion is further enhanced in the displayed image at block 614. In a preferred arrangement, the data in a volume of voxels is collected during a progression of pulse sequences which image the volume as a unit rather than as a series of slices. The spatial resolution of the image becomes progressively finer as the duration of each imaging pass increases as more data values are received.

In an arrangement that is particularly apt for breast imaging, a spiral “RODEO” imaging technique is employed. The acronym “RODEO” is for rotating delivery of excitation off resonance. In a RODEO spiral three dimensional imaging process, gradient field modulation is arranged for the acquisition of voxel values in a spiral that traverses k-space in the imaging plane. An RF pulse is used together with gradient fields that define a spiral sequence for excitation and detection. The preferred RF pulse excites only protons in water molecules resulting in fat-suppressed images. The particular pulse sequence quickly produces T1-weighted images that proceed in a spiral. Maintaining biasing field (B₀) homogeneity across the imaging FOV during spiral scanning helps to produce a high-resolution image. Tight specifications on shimming and eddy current compensation are also preferred to produce the desired image resolution. A two-dimensional (2D) Fourier Transform is applied to the data along a spiral trajectory through K-space. The object image is reconstructed from the spirally-progressing MRI signal.

In the pulse sequence design (i.e., the planned timing and sequence of excitation and gradient pulses), a slew rate-limited spiral trajectory gradient waveform is generated and applied repetitively in multiple shots, with variations of the spiral pitch or centering of the pattern. Varying the spiral pitch progressively fills in the k-space data enabling the generation of an image with a finer resolution. In a preferred sequence, a multiple-shot interleaved spiral trajectory is implemented. In the multiple-shot spiral sequence, each spiral can have fewer turns with a widened gap between the turns. The missing data in the widened gap is then filled in using additional spiral shots. The additional or subsequent spirals can have the same number of turns as the preceding spiral(s), but with a rotated trajectory in the k-space plane.

A multi-shot spiral data collection sequence with incrementally displaced (e.g., rotated) trajectories in k-space is advantageous over a single-shot technique. Although multi-shot spiral imaging generally requires a longer scan time than single-shot spiral imaging, the multi-shot spiral collection sequence obtains a greater level of detail than a single-shot technique as the image resolution is built up over the multiple shots. Additionally, the readout time required for the multiple shots is minimal which helps moderate off-resonance effects. Also, the spiral imaging technique is less demanding on the slew rate when compared to the single-shot spiral technique. The multi-shot, interleaved trajectory is implemented by rotating a matrix multiplier in the pulse sequence programming.

A spiral trajectory in k-space generally is defined by:

k=λθe^{iθ}

Where, k(t)=kₓ(t)+ik_{y}(t) is the complex location in k-space, and λ=Nᵢₙₜ/(2πFOV), Nᵢₙₜ is the number of interleaves, FOV is the field of view, and θ(t) is a function of time t to be defined.

By definition, the gradient is given by

$$g = \frac{1}{γ} \frac{d k}{d t}$$

Where, g(t)=gₓ(t)+ig_{y}(t) is the complex gradient waveform.

In this design, a slew rate-limited solution of θ(t) is used to generate the gradient waveform. For a given allowed slew rate, S₀, a gradient amplitude-limitation is applied. In particular, a maximum gradient of the waveform is checked against the maximum allowed gradient, G₀, as defined in scanner's system specifications.

A software waveform generator can be applied as a preliminary step to pre-calculate the gradient waveforms in iterations that are stored and read out during imaging rather than being repetitively generated. The gradient waveforms and trajectories in k-space that are produced and stored are used in both a pulse sequence application and in image reconstruction. Shifting the entire spiral trajectory by k_{c} in k-space helps reduce the impact of distortion in the k-space sampling location. This technique may be implemented by applying a constant unipolar gradient on both Gₓ and G_{y} before the spiral gradients. The distance that the k-space center is been shifted is determined by k_{c}. In practice, k_{c} is about 5% of the diameter of the sampled region.

To meet gradient system constraints and at the same time reduce the potential for imaging artifacts, a multiple-shot interleaved spiral trajectory also is implemented. The base spiral gradient waveform is pre-calculated and saved in a waveform library in a memory that is accessible to the controller. The pulse sequence is provided by loading the base sequence from the library. From the base waveform, the physical gradients Gₓ and G_{y} can be rotated about the z-axis during sequence repetitions. The angle of rotation may start at zero and be incremented at an angle that depends on the desired number of interleaved shots, Nᵢₙₜ. For example, if four interleaved shots are desired, Nᵢₙₜ=4, then the rotation angle would be 90 degrees, as 360°/Nᵢₙₜ equals 90 degrees. However, each subsequent interleaved shot does not necessarily have to be offset at an angle equal to 360°/Nᵢₙₜ, as random offset angles may also be implemented.

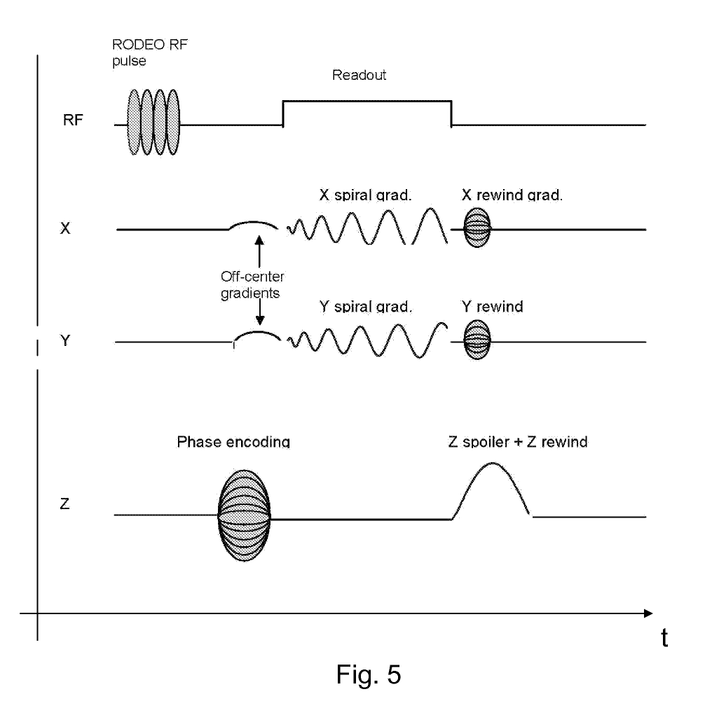

A preferred pulse sequence is shown in the timing diagram of FIG. 5. The pulse sequence consists of a RODEO RF pulse (described further below), followed by off-centering gradients to displace the current sensing position along the x- and y-axes, and a phase-encoding gradient that progresses along the z-axis. The specific spiral sequence in the x-y plane can be an Archimedes spiral, equiangular spiral, or another spiral form, provided that the collected data is interpreted to match the same spiral sequence and form. At the end of a readout, rewinding-gradient pulses are applied to all three axes to reset the nuclear spins. A spoiler-gradient pulse is applied along the z-axis and to desynchronize and randomize residual nuclear spins.

A preferred imaging sequence uses a RODEO RF pulse comprising two back-to-back cosine-shaped pulses. The first cosine shaped pulse, extends from 0 to 2π radians, and is centered on the resonance frequency of fat. This RF pulse is immediately followed by a similar cosine-shaped pulse having the same period, amplitude, and frequency as the first RF pulse, but phase-shifted 180 degrees. The combination of the two cosine-shaped, phase-reversed pulses results in the substantial cancellation of on-resonance spins thereby suppressing the fat-response signal in the collected data images. For off-resonance spins, the two RF pulses constructively interfere, resulting in an increased amplitude. Since water is off-resonance for the two cosine-shaped pulses, features within the patient's body having a high-water content are displayed with a higher contrast, and fatty tissues are suppressed.

The image reconstruction from the spiral k-data is implemented using an algorithm of non-uniform Fast Fourier Transforms (“FFT”). This method generates a 2D gridding kernel matrix for a given spiral trajectory using a least squares approach. More specifically, the reconstruction process consists of the following steps:
applying a 1D FFT along the z-axis on acquired data;
generating the kernel matrices corresponding to the spiral trajectory;
gridding k-data by convolving spiral k-data with the kernel matrices;
performing filtering and a 2D FFT on gridded k-data; and
resealing and formatting the images.

A 1D FFT is applied in the slice direction for each of the two dimensional k-space data points. This process allows zero-fill upon reconstruction parameter request.

According to the foregoing description, the data points are collected as a set of points along lines parallel to the z-axis and are centered on x-y points that proceed in a spiral rather than in a rectilinear raster. Although it is generally convenient to aim for equally spaced voxel positions, it is not mandatory that the data points have an equal density throughout the volume. Therefore, options can be provided for non-uniform sampling re-gridding along this dimension in order to reduce redundancy and/or wrap-around artifacts.

As mentioned above, the x-y points of the spiral trajectory can be pre-calculated as a spiral trajectory in a Cartesian, kₓ and k_{y}, or other coordinate system where the x-y points define each data collection point in k-space. These coordinates can be saved in a text file that can be loaded by the processor 44 at a later time. In an exemplary embodiment, the gradient waveform used in the pulse sequence has the same trajectory to reduce potential rounding errors. The file of x-y coordinates can be loaded from a file name provided from the reconstruction parameter set and include variations in the file data.

Next, the kernel matrices p₁ and p₂ are generated. Matrix P1 corresponds to trajectory kₓ and matrix p₂ corresponds to trajectory k_{y}. The matrices are generated as follows:

$${ρ}_{j , {c}_{p}} = {G}_{j , k} {a}_{k , {c}_{p}} , \\ j , k = - \frac{q}{2} … \frac{q}{2} , p = 1 … M ,$$

where,
p is the index of the data on the k-space trajectory and M is the number of non-uniformly spaced k-space data points;
m represents the scaling factor of FOV;
q is an even number representing the window width used in the gridding process;
and
cₚ is a real number of either the kₓ or k_{y} value.

The matrices G and F are opposite sides of the transform: G=F⁻¹, and the elements of matrix F are:

$${F}_{j , k} = \frac{- 2 j sin ( π ( j - k ) / m ) }{1 - exp ( i 2 π ( j - k ) / mN )} , \\ {a}_{k , cp} = i {\sum }_{γ = - 1 , 1}^{ } \frac{sin [ \frac{π}{2 m} ( 2 k - γ - 2 \{ {mc}_{p} \} ) ]}{1 - exp ( i \frac{π}{Nm} ( 2 \{ {mc}_{p} \} - 2 k + γ ) )}$$

$$\{ {mc}_{p} \} = {mc}_{p} - [ {mc}_{p} ]$$

where, [mcₚ] denotes the integer nearest to mcₚ.

A density compensation function (DCF) can be applied to effectively produce a uniform k-space density in the collected data. The DCF is defined as

D(k)=|k′∥sin(arg{k′}−arg{k})|,

where k′ is the k-space velocity vector.

In a preferred embodiment, density correction is also utilized by convolving the density-corrected k-space data sₚDₚ and the kernel matrices ρ₁ and ρ₂ to obtain gridded k-space data τ(k1,k2). The convolution is as follows:

$$τ ( k 1 , k 2 ) = {\sum }_{[ {mk}_{xp} ] + {j}_{1} = k 1}^{ } {\sum }_{[ {mk}_{yp} ] + {j}_{2} = k 2} {s}_{p} {D}_{p} {ρ}_{1} ( {j}_{1} , {k}_{xp} ) {ρ}_{2} ( {j}_{2} , {k}_{yp} ) ,$$

where,

p=1, . . . , M, j₁, j₂=−q|2, . . . q/2.

p is the index of the data on the k-space trajectory; and

M is the number of k-space data points.

Each gridded frame is filtered and 2D FFT transformed to obtain images wherein the data values are mapped, for example, to incremental levels of luminance. The 2D FFT dimensions are of mN*mN on τ(k1,k2). The field of view of the reconstructed image at this stage is mFOV.

### EXEMPLARY EMBODIMENTS

In an exemplary embodiment, a magnetic resonance imaging system comprises a biasing field magnet and an array of gradient magnet fields, a radio frequency pulse source, and a radio frequency receiver. The magnetic resonance imaging system further includes a control system and a processor coupled to the radio frequency receiver. The control system is operable to apply a magnetic field via the biasing field magnet and the gradient magnet fields. The processor is programmed and operable to decode a k-space MRI image from a signal emitted from a sample to be placed in the magnetic field and subjected to the pulse sequence. The processor is further coupled to collect a set of plural successive images, wherein at least one of the successive images is a reference image that is non-spoiled and at least one other one of the successive images is a subject image preceded by phase spoiling. The processor is further operable to subtract at least a component of the non-spoiled reference image from said subject image to obtain an output image.

In some embodiments, the phase spoiling can be configured to randomize previously synchronized magnetic moments that precess in a volume of nuclei of the sample. The volume of the sample can be selected using the gradient magnet fields.

In some embodiments, the processor comprises a digital processor coupled to a memory that is operable to store a three dimensional array of voxel data that represents the sample. The processor also may include an arithmetic unit that numerically subtracts an array of voxel data for the reference image from an array of voxel data for the subject image in the registry with the reference image to obtain the output image.

In some embodiments, the magnetic imaging system may further include a display system that is coupled to the memory and to the processor. The display system may be operable to selectively display an output image, the reference image, and the subject image.

In some embodiments, the processor and/or the display may be configured to distinguish a predetermined tissue type in the voxel data of the reference image. The predetermined tissue type may then be color coded in the reference image, the output image, or both the reference image and the output image.

In some embodiments the control system, radio receiver, and processor are configured to collect the plural successive images. The collection of the plural successive images is collected using either Cartesian or spiral image slice trajectory.

In an exemplary embodiment, a magnetic resonance imaging process includes the steps of placing a sample in a magnetic field established by a biasing field magnet and an array of gradient magnet fields, and applying a magnetic field and pulse sequence to the sample. The magnetic field being applied via the biasing field magnet and the gradient magnet fields and the pulse sequence applied via a radio frequency pulse source. The method further includes receiving a responsive radio frequency signal via a radio frequency receiver and decoding a k-space MRI data emitted from the sample. The applying, receiving, and collecting steps may be repeated and modified to include phase spoiling thereby obtaining one or more subject images. The magnetic resonance imaging process further includes subtracting at least a component of the non-spoiled reference image from the subject image to obtain an output image.

In some embodiments, the method further comprises the step of applying a contrast agent to the sample. The reference image is collected from the sample prior to the application of the contrast agent and the subject image is collected from the sample subsequent to the application of the contrast agent.

In some embodiments, the method further includes the step of collecting a succession of subject images during the wash in and out of the contrast agent in the sample.

In some embodiments, the reference image has a relatively higher gain with respect to fluid and edema when the contrast agent has an affinity for lesions, such as gadolinium-based contrast agents, so that subtracting the component of the reference image enhances the visibility of the lesions. In some embodiments, the full reference image is subtracted from the subject image.

In some embodiments, the method further includes the step of color coding concentrations of at least one composition in the output image, or an element of such compositions, for example color coding water concentrations to highlight edema and cysts.

In some embodiments, the phase spoiling pulse is configured to randomize previously synchronized magnetic moments that precess in a volume of nuclei of the sample. The volume of nuclei in the sample can be selected using the gradient magnet fields.

The disclosed technique is applicable to identify distinctions in various materials, not limited to tissue types with water versus fat concentrations, but also including highlighting of other pertinent compositions. An advantageous embodiment, for example, is color coding an image to identify volume areas containing concentrations of silicone, namely breast implant material. In this embodiment, an additional image data set is acquired wherein the silicone response signal is suppressed. That is, the magnetic response of the associated molecule (or an atom in the molecule) is used to develop and to enhance a visible distinction in the image displayed to the practitioner or technologist. Subtraction of the silicone suppressed image from that of non-silicone suppressed image produces an image with highlighted areas that in a projection of the image identifies pixels corresponding to volume elements (voxels) with silicone present.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A magnetic resonance imaging system, comprising:
a biasing field magnet and an array of gradient magnet fields,
a radio frequency pulse source;
a radio frequency receiver;
a control system operable to apply a magnetic field via the biasing field magnet and the gradient field magnets and to trigger application of a pulse sequence via the radio frequency pulse source,
a processor coupled to the radio frequency receiver, wherein the processor is programmed and operable to decode a k-space MRI image from a signal emitted from a sample to be placed in the magnetic field and subjected to the pulse sequence, wherein the processor is programmed to collect a set of plural successive images, wherein at least one of the successive images is a reference image that is non-spoiled and at least one other one of the successive images is a subject image preceded by a phase spoiling pulse, and wherein the processor is operable to subtract at least a component of the non-spoiled reference image from said subject image to obtain an output image.

2. The magnetic resonance imaging system of claim 1, wherein the phase spoiling pulse is configured to randomize previously synchronized magnetic moments precessing in a volume of nuclei of the sample as selected using the gradient field magnets.

3. The magnetic resonance imaging system of claim 1, wherein the processor comprises a digital processor coupled to a memory operable to store at least one three dimensional array of voxel data representing the sample, and an arithmetic unit numerically to subtract an array of the voxel data for the reference image from an array of voxel data for the subject image in registry with the reference image, to obtain said output image.

4. The magnetic resonance imaging system of claim 3, further comprising a display system coupled to the memory and to the processor, wherein the display system is operable selectively to display at least one of the output image, the reference image and the subject image.

5. The magnetic resonance imaging system of claim 4, wherein at least one of the processor and the display is configured to distinguish a predetermined tissue type in the voxel data of the reference image and to color code said predetermined tissue type in at least one of the reference image and the output image.

6. The magnetic resonance imaging system of claim 4, wherein at least one of the processor and the display is configured to distinguish a predetermined composition in the voxel data of the reference image and to color code said predetermined composition in at least one of the reference image and the output image.

7. The magnetic resonance imaging system of claim 6, wherein the predetermined composition that is distinguished comprises at least one of water, fat, a contrast agent, silicone, and at least one element contained therein.

8. The magnetic resonance imaging system of claim 2, wherein the control system, radio receiver and processor are configured to collect said plural successive images using a spiral image slice trajectory.

9. A magnetic resonance imaging process comprising the steps of:
placing a sample in a magnetic field established by a biasing field magnet and an array of gradient field magnets,
applying a magnetic field via the biasing field magnet and the gradient field magnets and applying a pulse sequence to the sample via a radio frequency pulse source,
receiving a responsive radio frequency signal via a radio frequency receiver and decoding a k-space MRI image emitted from the sample;
collecting from the sample at least one image, wherein the pulse sequence does not employ phase spoiling, thereby obtaining a non-spoiled reference image of the sample;
repeating said applying, receiving and collecting steps wherein the pulse sequence is modified to include phase spoiling, thereby obtaining at least one subject image;
subtracting at least a component of the non-spoiled reference image from said subject image to obtain an output image.

10. The process of claim 9, further comprising applying a contrast agent to the sample, and wherein the reference image is collected from the sample prior to application of the contrast agent and the subject image is collected from the sample subsequent to application of the contrast agent.

11. The process of claim 10, further comprising collecting a succession of subject images during diffusion of the contrast agent in the sample.

12. The process of claim 11, wherein the reference image has a relatively higher gain with respect to fluid and edema and wherein the contrast agent has an affinity for lesions, whereby subtracting the component of the reference image enhances visibility of said lesions.

13. The process of claim 11, wherein the contrast agent comprises gadolinium.

14. The process of claim 12, wherein the reference image is subtracted in full from the subject image.

15. The process of claim 10, further comprising color coding at least one of the fluid and edema in the output image.

16. The process of claim 10, wherein the phase spoiling pulse is configured to randomize previously synchronized magnetic moments precessing in a volume of nuclei of the sample as selected using the gradient field magnets.

17. The process of claim 10, further comprising color coding the output image to highlight a predetermined composition.

18. The process of claim 17, wherein the predetermined composition that is highlighted comprises at least one of water, fat, a contrast agent, silicone, and at least one element contained therein.
